(19) United States
(12) Patent Application Publication
(10) Pub. No.: US 2024/0415559 A1
(43) Pub. Date: Dec. 19, 2024

(54) MICROPHONE BRUIT SENSING FOR CRYOABLATION BALLOON CATHETERS

(71) Applicant: Medtronic Ireland Manufacturing Unlimited Company, Dublin 2 (IE)

(72) Inventor: Binit Panda, Maple Grove, MN (US)

(21) Appl. No.: 18/738,238

(22) Filed: Jun. 10, 2024

Related U.S. Application Data
(60) Provisional application No. 63/508,122, filed on Jun. 14, 2023.

(51) Int. Cl. A61B 18/02; A61B 18/00; A61M 25/10

(52) U.S. Cl. CPC A61B 18/02; A61M 25/10181; A61B 2018/0022; A61B 2018/00642; A61B 2018/00744; A61B 2018/00863; A61B 2018/0212; A61M 2205/0294; A61M 2205/3334; A61M 2205/3375

(57) ABSTRACT

Devices, systems, and methods for performing cryoablation using a single sized balloon coupled with one or more audio sensors for use in controlling balloon inflation. One example system comprises an electronic controller configured to couple to a balloon catheter comprising a balloon, an inflow lumen for providing a fluid to the balloon, and a microphone sensor is positioned to sense a flow of blood proximal to the balloon. The electronic controller is configured to receive, from the microphone sensor, a signal indicative of a blood flow sound. The electronic controller is configured to control a pump to provide a volume of the fluid to the balloon via the inflow lumen based at least in part on the signal indicative of the blood flow sound.

### RELATED APPLICATIONS

This application claims the benefit of U.S. Provisional Application Ser. No. 63/508,122, filed Jun. 14, 2023, the entire contents of each of which are incorporated herein by reference.

### BACKGROUND

Cryoablation therapy is used to treat a number of conditions, including uncontrolled hypertension and cardiac arrhythmias, for example, through vascular denervation. During vascular denervation, treatment elements on the distal end of a transcutaneous catheter deliver a pressurized refrigerant to a treatment area of a vessel to damage targeted nerves. The flow of refrigerant causes an occlusive balloon of the treatment element to expand within the vessel. This occludes the vessel while thermal transfer from the tissue to the refrigerant creates a circumferential lesion at the treatment site.

### SUMMARY

Some vascular denervation catheter devices use cryoablation techniques. During cryoablation treatment, a pressurized refrigerant is circulated through an occlusive balloon, which has been inserted into a patient's vessel. The flow of refrigerant causes the occlusive balloon to expand within the vessel. Adequate balloon expansion must be achieved and maintained to hold the occlusive balloon in place during the treatment, thus ensuring complete occlusion of the vessel (and thus good thermal contact between the occlusive balloon and the vessel wall) and creation of a circumferential lesion at the treatment site.

Balloon catheters are traditionally designed to target a particular vessel size at a given pressure. Some denervation cryocatheters feature a single, semi compliant or compliant balloon for use in denervating vessels spanning from 3 mm to 8 mm with a single balloon. Such devices may have a nominal expansion diameter of 8 mm at approximately 20 psi. To allow use over diametrical ranges, some catheter balloons include a number (e.g., from 3-6) balloon pleated folds, which unfold as the balloon expands.

Because these single balloons cover large diametrical ranges, the coverage and contact from the balloon to the vessel is not optimized for each single diameter. Rather, internal balloon pressure is relied upon to create the contact against the wall of the vessel. To reduce the risk of overdilation of the renal vessel under treatment, it the force per unit area on the vessel may be controlled. Radial force increases at double the rate of the pressure. It is therefore desirable to use the lowest effective pressure during treatment. Some treatment methods use a flow wire in the vessel or track balloon inflation with fluoroscopy. However, a flow wire uses the doppler principle and needs to transmit a signal to receive the reflection. This requires adding complex ultrasonic sensors to the flow wire. While fluoroscopy may provide a good confirmation whether or not the occlusion is 100%, is may not be able to indicate whether the occlusion is anything between 50-90%. In addition, a high amount of contrast is needed to monitor occlusion using fluoroscopy.

To address these issues, devices, systems, and methods are provided herein for performing cryoablation using a single sized balloon coupled with one or more audio sensors for use in controlling balloon inflation. Embodiments and aspects described herein provide, among other things, balloon catheters with treatment elements that include a microphone sensor, which detect vascular sounds caused by turbulent blood flow (bruits) through an area of the vessel under treatment. Because bruits can be related to a degree of vessel occlusion by the balloon, embodiments and aspects described herein use microphone sensors detect bruits and control catheter balloon inflation based on analysis of the bruits.

Unlike ultrasonic systems, the examples provided herein require only a passive measurement element, and need not account for doppler properties and transmission of any ultrasound signal. In addition, a transducer assembly of a microphone sensor is simpler than an ultrasonic sensor.

In the embodiments and aspects described herein, the degree of occlusion can be calculated in real-time or near real-time, which can enable a controller to implement a closed-loop balloon expansion mechanism using the bruits. In some aspects, microphones may be placed at multiple locations, e.g., proximally and distally to the balloon. As the balloon expands, the blood flow is occluded, which will change the hemodynamics of the flow. Sensors proximal or distal to the balloon are able to detect the difference in the hemodynamics.

Examples and aspects provided herein are described with respect to cryoablation and denervation. However, this should not be considered limited. In some aspects, the examples presented herein are applicable to any type of balloon catheter, e.g., an angioplasty balloon catheter, a stent delivery balloon catheter, and the like.

In some aspects, the techniques described herein relate to a system. The system includes an electronic controller. The electronic controller is configured to couple to a balloon catheter comprising a balloon, an inflow lumen for providing a fluid to the balloon, and a microphone sensor is positioned to sense a flow of blood proximal to the balloon. The electronic controller is configured to receive, from the microphone sensor, a signal indicative of a blood flow sound.

The electronic controller is configured to control a pump to provide a volume of the fluid to the balloon via the inflow lumen based at least in part on the signal indicative of the blood flow sound.

In some aspects, the techniques described herein relate to a method. The method includes receiving, from a microphone sensor positioned to sense a flow of blood proximal to a balloon of a balloon catheter, a signal indicative of a blood flow sound. The method includes controlling a pump to provide a volume of fluid to the balloon based at least in part on the signal indicative of the blood flow sound.

In some aspects, the techniques described herein relate to a balloon catheter. The balloon catheter includes a balloon, an inflow lumen configured to provide a fluid to the balloon, and a microphone sensor. The microphone sensor is coupled to the elongate body proximal of the balloon. The microphone sensor is configured to sense a flow of blood proximal to the balloon.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments, examples, aspects, and features of concepts that include the claimed subject matter and explain various principles and advantages of those embodiments, examples, aspects, and features.

FIG. 1 is schematic illustration of a vascular denervation system according to some examples.

FIG. 2 illustrates a treatment element of the vascular denervation system of FIG. 1 according to some examples.

FIG. 3 is a block diagram that illustrates an electronic controller of the system of FIG. 1 according to some examples.

FIG. 4 is a flow chart illustrating a method for operating the system of FIG. 1 according to some examples.

FIG. 5 is a flow chart illustrating a method for operating the system of FIG. 1 according to some examples.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of examples, aspects, and features illustrated.

In some instances, the apparatus and method components have been represented where appropriate by conventional symbols in the drawings, showing only those specific details that are pertinent to understanding the of various embodiments, examples, aspects, and features so as not to obscure the disclosure with details that will be readily apparent to those of ordinary skill in the art having the benefit of the description herein.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Also, it is to be understood that the phraseology and terminology used herein is for the purpose of description and should not be regarded as limiting. The terms “mounted,” “connected,” and “coupled” are used broadly and encompass both direct and indirect mounting, connecting, and coupling. The terms “connected” and “coupled” are not restricted to physical or mechanical connections or couplings, and can include electrical connections or couplings, whether direct or indirect. Electronic communications and notifications described herein may be performed using any known or future-developed means including wired connections, wireless connections, etc.

For ease of description, some or all of the example systems presented herein are illustrated with a single exemplar of each of its component parts. Some examples may not describe or illustrate all components of the systems. Other embodiments may include more or fewer of each of the illustrated components, may combine some components, or may include additional or alternative components.

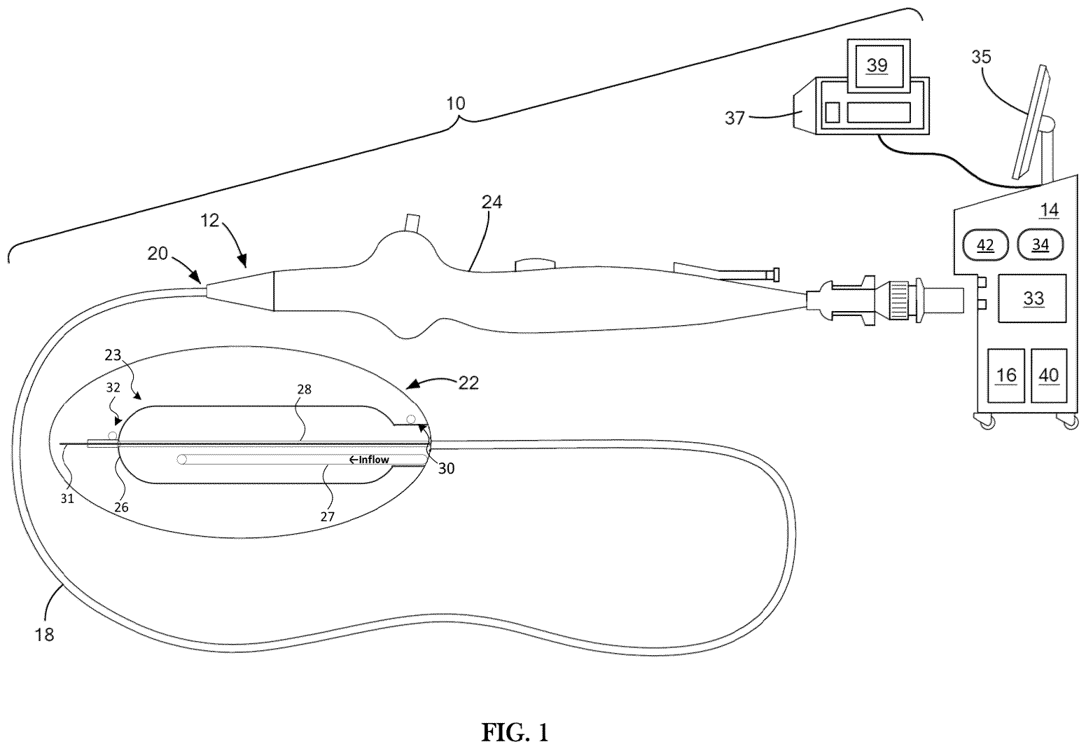

FIG. 1 illustrates an example system 10 that is suitable for performing cryoballoon ablation, including denervation. The system 10 is configured to perform denervation in the renal arteries, as well as in other vessels, including the celiac trunk and its branches, the splenic artery and its branches, the common hepatic artery and its branches, the left gastric artery and its branches, and the superior and inferior mesenteric arteries and their branches. The system 10 may also be configured to perform denervation two or more vessels (e.g., in the renal artery and the common hepatic artery and its branches).

The system 10 is for delivering thermally conductive fluid (refrigerant) to an arca of target tissue, such as, for example, an area of tissue within the hepatic artery to cause denervation. The system 10 may generally include a catheter, such as a catheter 12, and a console 14 for operating, monitoring, and regulating the operation of the catheter 12 (for example, with the electronic controller 33), and a fluid source 16 for delivering fluid (for example, a refrigerant) to the catheter 12.

The catheter 12 is a highly flexible treatment device that is suitable for passage through the vasculature. The catheter 12 may be adapted for use with the fluid source 16 to denervate portions of an artery. In the example illustrated, the catheter 12 has an elongate body 18 having a proximal portion 20 and a distal portion 22. The distal portion 22 includes a treatment element 23. The proximal portion 20 of the catheter 12 is mated to a handle 24 that can include an element such as a lever or knob for manipulating the elongate body 18 and the treatment element 23. The distal portion 22 may also include an aperture (not shown) sized to allow for the passage of a guidewire 31 through the elongate body 18 and through the aperture.

The elongate body 18 is sized and configured to be passable through a patient's vasculature and/or positionable proximate to the area of target tissue, and may include one or more lumens (for example, the inflow lumen 27 and the inner member/guidewire lumen 28) disposed within the elongate body 18 that provide mechanical, electrical, and/or fluid communication between the proximal portion 20 of the elongate body 18 and the distal portion 22 of the elongate body 18. In some aspects, the elongate body 18 or a portion thereof includes a guidewire lumen through which a sensing device, mapping device, the guidewire 31, or other system components may be located and extended from the distal portion 22 of the catheter 12. Thus, catheter 12 may be an over-the-wire (OTW) or rapid exchange (RX) design. The elongate body 18 may be rigid and/or flexible to facilitate the navigation of the catheter 12 within a patient's body. In one aspect, the distal portion 22 of the elongate body 18 is flexible to allow for more desirable positioning proximate to an arca of target tissue (e.g., positioning within a renal artery, a hepatic artery, the splanchnic bed, the pulmonary artery, the aortic root, the carotid body, and the like). To access an area of target tissue, the catheter 12 may be inserted through one or more blood vessels, such as, for example, one or more brachial arteries, one or more radial arteries, one or more femoral arteries, or other points of access including venous access (e.g., for greater splanchnic nerve denervation or through the jugular vein for carotid body ablation).

The treatment element 23 includes a balloon 26 (for example, an occlusive balloon), through which fluid from the fluid source 16 is circulated. An inflow lumen 27 is in fluid communication with the fluid source 16 in the console 14 to supply a refrigerant fluid (e.g., nitrous oxide (N₂O), nitrogen, carbon dioxide, argon, or another suitable refrigerant) in response to console commands and other control input. In some aspects, a vacuum pump 34 (electronically coupled to and controlled by the electronic controller 33) in the console 14 creates a low pressure environment in an outflow lumen (not shown) so that the fluid is drawn into the outflow lumen, away from the balloon 26, towards the proximal portion 20 of the elongate body 18, and into the fluid recovery reservoir 40 within the console 14. In some aspects, the electronic controller 33 controls a position of a valve or valves (not shown) to control the flow of a refrigerant fluid (e.g., stored under pressure in the fluid source 16) toward the treatment element 23. When expanded, the balloon 26 is sized and configured to fit within an area of the artery under treatment such that the balloon 26 is substantially centered within the artery. In some examples, the balloon 26 is a single sized 8 mm compliant balloon for in vessel ranges from 3-8 mm (e.g., renal main arteries, common hepatic arteries, or the like). Aspects presented herein are also applicable to other types (e.g., sizes) of occlusive balloons.

The treatment element 23 also includes one or more microphone sensors, such as a first microphone sensor 30 and/or a second microphone sensor 32. In the illustrated example, the first microphone sensor 30 is positioned proximally to the balloon 26 and the second microphone sensor 32 is positioned distally to the balloon 26. The first microphone sensor 30 and the second microphone sensor 32 are transducers configured to sense sounds produced within a vessel under treatment (e.g., by the flow of blood through the vessel). The microphone sensors 30, 32 are coupled to the electronic controller 33 of the console 14, e.g., by one or more wires extending within elongate body 18. In some aspects, the microphone sensors 30, 32 are directly coupled to the electronic controller 33, which generates audio from voltages or other signals read from or provided by the microphone sensors 30, 32. In other aspects, the microphone sensors 30, 32 are indirectly coupled to the electronic controller 33 through, for example, intervening circuitry in the catheter 12 and the electronic controller 33 receives audio from the intervening circuitry. In some aspects, the first microphone sensor 30 and the second microphone sensor 32 are affixed (e.g., with a glue) to the catheter 12, e.g., are coupled to the elongate body 18.

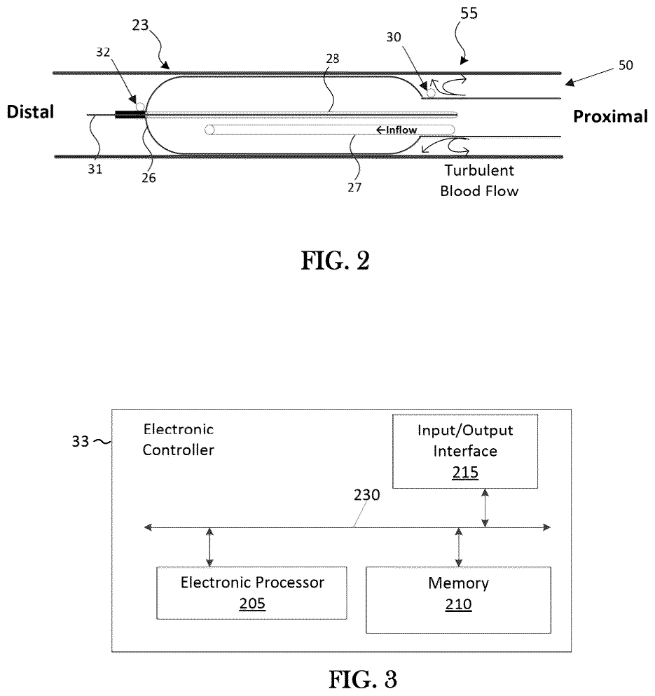

FIG. 2 illustrates the treatment element 23 while deployed in an artery 50. As illustrated in FIG. 2, as the balloon 26 expands within the artery 50, blood flow through the artery 50 is reduced and a turbulent blood flow 55 is produced proximal and/or distal of the balloon 26. As described herein, the first microphone 30 is positioned to sense the sounds made by the turbulent blood flow 55 proximal to the balloon 26, while the second microphone 32 is positioned to sense the sounds made by any blood that flows past the balloon 26 distally.

In the example illustrated in FIGS. 1 and 2, the treatment element 23 includes two microphone sensors positioned outside of the balloon 26. In other examples, the microphone sensors may be positioned within the balloon 26. In another example, the treatment element includes only one microphone sensor, positioned proximally to the balloon 26. In another example, the treatment element includes more than two microphone sensors.

Returning to FIG. 1, in some aspects, the console 14 includes one or more pressure sensors 42 to continuously record the instantaneous pressure values within the balloon 26. The pressure sensors 42 may then generate and transmit a pressure signal to the electronic controller 33 of the console 14. In some aspects, treatment element 23 also includes a pressure monitoring tube (enclosed in the elongate body 18) in fluid communication with the pressure sensor 42 (housed in the console 14) and the balloon 26.

In the illustrated example, the control unit 14 includes an electronic controller 33 (described more particularly with respect to FIG. 3) programmed or programmable to execute the automated or semi-automated operation and performance of the features, sequences, calculations, or procedures described herein. The electronic controller 33 is communicatively coupled to the various components of the control unit 14, including the vacuum pump 34, one or move valves as described herein, and various components of the catheter 12, such as the first microphone sensor 30 and the second microphone sensor 32. The control unit 14 may include one or more user input devices, controllers, speakers, and/or electronic displays 35 (each coupled to and controllable by the electronic controller 33) for collecting and conveying information from and to the user.

In some aspects, the treatment element 23 includes ultrasonic transducers (not shown) to record the reflected, refracted, scattered, and/or attenuated ultrasound signals from the target tissue. As the transducers record the ultrasound signals, they vibrate and the mechanical vibrations are converted into electric current signals that are transmitted back to the control unit 14 or an external ultrasound control unit 37 (FIG. 1) which processes the signals to generate a sonogram or ultrasonogram showing the patient's tissue, organs, and/or a location of the catheter 12 within the patient's body. The sonogram may then be relayed to a clinician via a display 35 of the control unit 14, or via a display 39 (FIG. 1) of the external ultrasound control unit 37, to assist a medical practitioner in positioning the catheter 12 at a desired treatment location.

FIG. 3 illustrates an example embodiment of the electronic controller 33, which includes an electronic processor 205 (for example, a microprocessor, application specific integrated circuit, etc.), a memory 210, and an input/output interface 215. The electronic processor 205, the memory 210, and the input/output interface 215, as well as the other various modules are coupled directly, by one or more control or data buses (e.g., the bus 230), or a combination thereof. The memory 210 may be made up of one or more non-transitory computer-readable media and includes at least a program storage area and a data storage area. The program storage area and the data storage area can include combinations of several types of memory, such as read-only memory (“ROM”), random access memory (“RAM”) (for example, dynamic RAM (“DRAM”), synchronous DRAM (“SDRAM”), etc.), electrically erasable programmable read-only memory (“EEPROM”), flash memory, or other suitable memory devices. The electronic processor 205 is coupled to the memory 210 and the input/output interface 215. The electronic processor 205 sends and receives information (for example, from the memory 210 and/or the input/output interface 215) and processes the information by executing one or more software instructions or modules, capable of being stored in the memory 210, or another non-transitory computer readable medium. The software can include firmware, one or more applications, program data, filters, rules, one or more program modules, and other executable instructions. The electronic processor 205 is configured to retrieve from the memory 210 and execute, among other things, software for performing methods as described herein.

The input/output interface 215 transmits and receives information from devices external to the electronic controller 33 (for example, over one or more wired and/or wireless connections), for example, components of the system 10. The input/output interface 215 receives input (for example, from a human machine interface of the console 14), provides system output or a combination of both. The input/output interface 215 may also include other input and output mechanisms, which for brevity are not described herein and which may be implemented in hardware, software, or a combination of both.

It should be understood that although FIG. 3 illustrates only a single electronic processor 205, memory 210, and input/output interface 215, alternative embodiments of the electronic controller 33 may include multiple processors, memory modules, and/or input/output interfaces. It should also be noted that the system 10 may include other electronic controllers, each including similar components as, and configured similarly to, the electronic controller 33. In some embodiments, the electronic controller 33 is implemented partially or entirely on a semiconductor (for example, a field-programmable gate array [“FPGA”] semiconductor) chip. Similarly, the various modules and controllers described herein may be implemented as individual controllers, as illustrated, or as components of a single controller. In some aspects, a combination of approaches may be used.

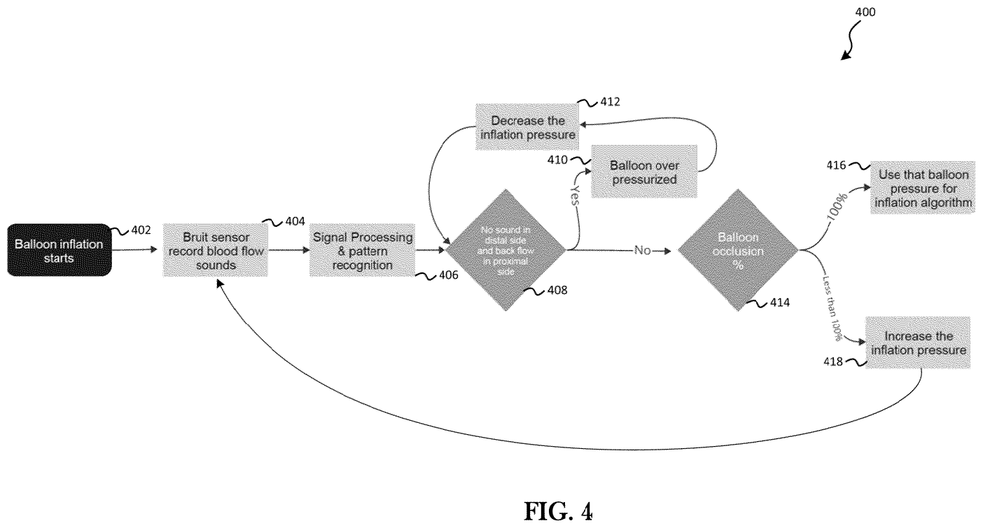

FIG. 4 illustrates an example method 400 for operating the system of FIG. 1 to monitor aspects of the balloon 26, through the use of either or both of the first and second microphone sensors 30, 20, to detect a degree of vessel closure achieved by the balloon during cryoablation treatment. Although the method 400 is described in conjunction with the system 10 as described herein, the method 400 could be used with other systems and devices. In addition, the method 400 may be modified or performed differently than the example provided.

As an example, the method 400 is described as being performed by the electronic controller 33. However, it should be understood that, in some examples, portions of the method 700 may be performed by other components, including for example, the catheter 12.

The method begins at block 402, with the treatment element deployed in an artery (e.g., the artery 50). At block 402, the electronic controller 33 initiates an ablation process, for example, by controlling the fluid source 16 to provide a volume of the fluid to the balloon 26 via the inflow lumen 27. In one aspect, the electronic controller 33 controls the vacuum pump 34 or a similar pump to provide a volume of refrigerant to the balloon 26. In another aspect, the electronic controller 33 controls a valve or valves to provide a volume of refrigerant to the balloon 26 from a pressurized source.

At block 404, one or more bruit sensors (e.g., the first microphone sensor 30, the second microphone sensor 32, or both) record blood flow sounds from the artery (55) under treatment. The electronic controller 33 receives the audio from the bruit sensors.

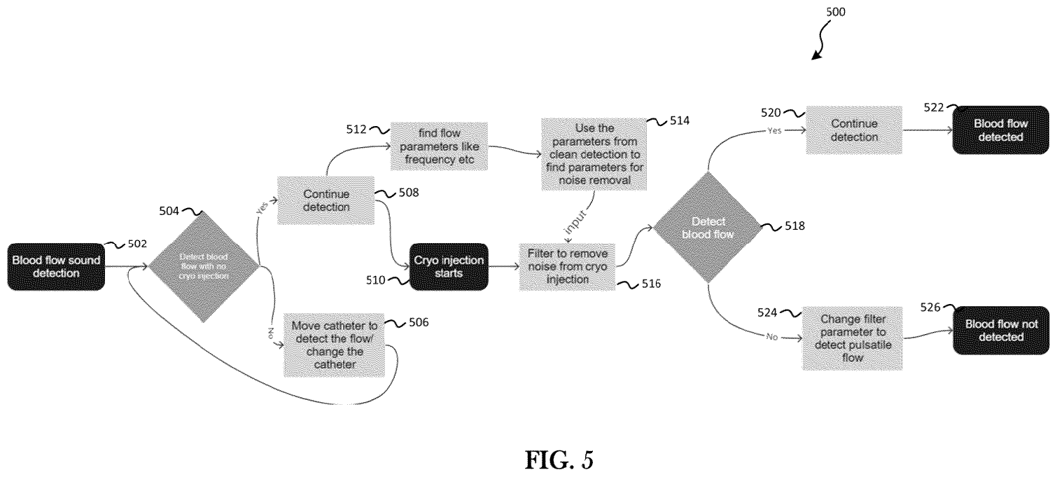

At block 406, the electronic controller 33 performs signal processing and pattern recognition on the recorded audio. One example method for such signal processing is described herein with respect to FIG. 5. In some embodiments, the process described with respect to blocks 502-522 of FIG. 5 is performed between blocks 404 and 406 of the method 400.

At block 408, the electronic controller 33 analyzes the results of the signal processing and pattern recognition to determine whether there is both no sound on the distal side of the balloon 26 and a back flow on the proximal side of the balloon 26.

If both conditions are true, the electronic controller 33 determines (at block 410) that the balloon is over pressurized and decreases the inflation pressure (at block 412). For example, the electronic controller 33 may control the fluid source to stop providing the volume of the fluid to the balloon 26 (e.g., stopping or reducing the fluid flow). In some aspects, the electronic controller 33 will also present an indication to an operator of the system, for example, indicating that the balloon is over pressurized and that deflation is occurring. In one example, the electronic controller 33 displays on the electronic display 35 a message to the operator stating “PRESSURE TOO HIGH. AUTO PRESSURE REDUCTION ACTIVATED.” In another example, the electronic controller 33 presents the indication by playing an audio message, activating a warning light, sounding an audio alarm, activating a haptic feedback motor, or performing some combination of the foregoing.

At block 414, when the electronic controller 33 determines (at block 408) that there is both sound on the distal side of the balloon 26 and a back flow on the proximal side of the balloon 26, it determines an occlusion percentage. For example, the electronic controller 33 may compare the blood flows distal and proximal to the balloon 26 to determine the occlusion percentage.

At block 416, when the electronic controller 33 determines (at block 408) that the occlusion percentage is 100%, the electronic controller 33 identifies the current pressure for the balloon 26 (e.g., using the pressure sensor 42) and uses the current pressure for the inflation algorithm use during treatment. For example, the electronic controller 33 controls the fluid source to maintain that pressure for the cryoablation treatment duration.

At block 418, when the electronic controller 33 determines (at block 408) that the occlusion percentage is less than 100%, the electronic controller 33 controls the fluid source to increase the pressure of the balloon 26 and continues to sense sounds and assess the occlusion percentage for the balloon 26 (at blocks 404-418).

FIG. 5 illustrates an example method 500 for operating the system of FIG. 1 to monitor aspects of the balloon 26, through the use of either or both of the first and second microphone sensors 30, 20, to detect blood flows during cryoablation treatment. Although the method 500 is described in conjunction with the system 10 as described herein, the method 500 could be used with other systems and devices. In addition, the method 500 may be modified or performed differently than the example provided.

As an example, the method 500 is described as being performed by the electronic controller 33. However, it should be understood that, in some examples, portions of the method 700 may be performed by other components, including for example, the catheter 12. In some aspects, the method 500 is performed by the electronic controller 33 while performing the method 400, as described above.

At block 502, the electronic controller 33 detects sounds from the artery (55) using one or more bruit sensors (e.g., the first microphone sensor 30, the second microphone sensor 32, or both).

At block 504, the electronic controller 33 determines whether blood flow sounds are detected while no cryo fluid is being delivered to the balloon 26. At block 506, when no blood flow sounds are detected (at block 504), the electronic controller 33 presents an indication to an operator of the system, for example, indicating that the operator should move the catheter or change the catheter. In one example, the electronic controller 33 displays on the electronic display 35 a message to the operator stating “BLOOD FLOW NOT SENSED. CHECK CATHETER.”

At block 508, when blood flow sounds are detected (at block 504), the electronic controller 33 continues detecting (i.e., receiving audio from the first microphone sensor 30, the second microphone sensor 32, or both) blood flow while cryo fluid is delivered to the balloon 26 (at block 510) and flow parameters are determined (at block 512). Flow parameters include, for example, a frequency. In another example, the flow parameter includes blood flow spectrum mean power and centroid and is measured by calculating the spectrum of the blood flow and finding the area of interest, which could be a frequency window, and calculating features such as mean power and centroid at that frequency window.

At block 514, the parameters determined at block 512 are used find parameters for noise removal. In some aspects, the noise is detected by finding outliers and sporadic spectrum peaks in the audio that are not related to a blood flow characteristic.

At block 516, the electronic controller 33 uses the parameters determined at block 514 to perform noise filtering on the audio collected during cryo fluid delivery. At block 518, the electronic controller 33 analyzes the noise-filtered audio to detect blood flow. For example, the electronic controller 33 may employ a pattern recognition algorithm to detect the sound of blood flows and turbulent blood flows from the audio. In some aspects, the electronic controller 33 analyzes the noise-filtered audio to detect blood pulsatile flow using the flow parameters described herein.

At block 520, where blood flow has been detected (at block 518), the electronic controller 33 continues detection and indicates that blood flow has been detected (at block 522). For example, the electronic controller 33 may indicate blood flow detection on a screen of the console 14 or may set a flag (e.g., for use by the method 400).

At block 520, where blood flow has not been detected (at block 518), the electronic controller 33 changes the filters (used at block 516) to detect a pulsatile flow and indicates that blood flow has not been detected (at block 526). Filter features control the area of interest detection, as described above with respect to block 508. The area of interest can be calculated by either having a stored area of interest that the algorithm compares to, or by dynamically detecting it by using machine learning and blood flow features such as BP, MAP, and the like.

For example, the electronic controller 33 may indicate no blood flow detection on a screen of the console 14 or may set a flag (e.g., for use by the method 400).

In the foregoing specification, specific embodiments are described. However, one of ordinary skill in the art appreciates that various modifications and changes may be made without departing from the scope of the invention as set forth in the claims below. Accordingly, the specification and figures are to be regarded in an illustrative rather than a restrictive sense, and all such modifications are intended to be included within the scope of present teachings. For example, while some embodiments are illustrated and described as including a single ultrasonic energy source, such embodiments could be applied to any balloon-based system, including those with other types or quantities of heating element energy sources.

It should also be noted that a plurality of hardware and software-based devices, as well as a plurality of different structural components may be utilized to implement the embodiments provided herein. It should also be noted that a plurality of hardware and software-based devices, as well as a plurality of different structural components may be used to implement the invention. In addition, it should be understood that embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software (e.g., stored on non-transitory computer-readable medium) executable by one or more processors. As such, it should be noted that a plurality of hardware and software-based devices, as well as a plurality of different structural components may be utilized to implement the invention. For example, “control units” and “controllers” described in the specification can include one or more processors, one or more application specific integrated circuits (ASICs), one or more memory modules including non-transitory computer-readable media, one or more input/output interfaces, and various connections (e.g., a system bus) connecting the components.

It will be appreciated that some embodiments may be comprised of one or more electronic processors such as microprocessors, digital signal processors, customized processors, and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, some embodiments may be implemented as a computer-readable storage medium having computer readable code stored thereon for programming a computer (for example, comprising an electronic processor) to perform a method as described and claimed herein. Examples of such computer-readable storage media include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

It should be understood that although certain drawings illustrate hardware and software located within particular devices, these depictions are for illustrative purposes only. In some examples, the illustrated components may be combined or divided into separate software, firmware and/or hardware. For example, instead of being located within and performed by a single electronic processor, logic and processing may be distributed among multiple electronic processors. Regardless of how they are combined or divided, hardware and software components may be located on the same computing device or may be distributed among multiple different devices.

In this specification, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a,” “has . . . a,” “includes . . . a,” or “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially,” “essentially,” “approximately,” “about,” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. A device or structure that is “configured” in a certain way is configured in at least that way but may also be configured in ways that are not listed.

The following paragraphs provide various examples of the embodiments disclosed herein.
Example 1. A system. The system comprises an electronic controller configured to couple to a balloon catheter comprising a balloon, an inflow lumen for providing a fluid to the balloon, and a microphone sensor is positioned to sense a flow of blood proximal to the balloon. The electronic controller is configured to receive, from the microphone sensor, a signal indicative of a blood flow sound. The electronic controller is configured to control a pump to provide a volume of the fluid to the balloon via the inflow lumen based at least in part on the signal indicative of the blood flow sound.
Example 2. The system of Example 1, further including the balloon catheter and the pump.
Example 3. The system of Example 1, wherein the electronic controller is configured to: perform signal processing on the signal indicative of the blood flow sound to produce a processed blood flow sound; determine, based at least in part on the processed blood flow sound, a balloon occlusion percentage; and control the pump to provide a volume of the fluid to the balloon via the inflow lumen based at least in part on the balloon occlusion percentage.
Example 4. The system of Example 3, wherein the electronic controller is configured to: when the balloon occlusion percentage meets a threshold, control the pump to maintain a current pressure in the balloon; and when the balloon occlusion percentage does not meet the threshold, control the pump to increase an inflation pressure for the balloon.
Example 5. The system of Example 1, wherein the electronic controller is configured to: perform signal processing on the signal indicative of the blood flow sound to produce a processed blood flow sound; determine, based at least in part on the processed blood flow sound, whether the balloon is over pressurized; and based at least in part on determining that the balloon is over pressurized, control the pump to decrease an inflation pressure for the balloon.
Example 6. The system of Example 1, wherein: the catheter includes a second microphone sensor positioned to sense a flow of blood distal to the balloon; and the electronic controller is further configured to: receive a second signal indicative of a second blood flow sound from the second microphone sensor; and control the pump to provide the volume of the fluid to the balloon via the inflow lumen based at least in part on the signal indicative of the blood flow sound and the second signal indicative of the second blood flow sound.
Example 7. The system of Example 6, wherein the electronic processor is configured to: determine whether the signal indicative of the blood flow sound indicates a back flow proximal to the balloon; determine whether the second signal indicative of the second blood flow sound indicates substantially no flow distal to the balloon; and responsive to determining that the signal indicative of the blood flow sound indicates a back flow proximal to the balloon and the second signal indicative of the second blood flow sound indicates substantially no flow distal to the balloon, control the pump to decrease an inflation pressure for the balloon.
Example 8. The system of Example 1, wherein the microphone sensor is a piezoelectric microphone.
Example 9. The system of Example 1, wherein the microphone sensor is positioned outside of the balloon.
Example 10. The system of Example 1, wherein the fluid is one selected from the group consisting of nitrous oxide, carbon dioxide, nitrogen, and argon.
Example 11. A method comprising: receiving, from a microphone sensor positioned to sense a flow of blood proximal to a balloon of a balloon catheter, a signal indicative of a blood flow sound; and controlling a pump to provide a volume of fluid to the balloon based at least in part on the signal indicative of the blood flow sound.
Example 12. The method of Example 11, further comprising: performing signal processing on the signal indicative of the blood flow sound to produce a processed blood flow sound; determining, from the processed blood flow sound, a balloon occlusion percentage; and controlling the pump to provide a volume of the fluid to the cryoablation balloon via the inflow lumen based on the balloon occlusion percentage.
Example 13. The method of Example 12, further comprising: when the balloon occlusion percentage meets a threshold, controlling the pump to maintain a current pressure in the cryoablation balloon; and when the balloon occlusion percentage does not meet the threshold, controlling the pump to increase an inflation pressure for the cryoablation balloon.
Example 14. The method of Example 12, further comprising: receiving a second signal indicative of a second blood flow sound from the second microphone sensor positioned to sense a flow of blood distal to the cryoablation balloon, and controlling the pump to provide the volume of the fluid to the cryoablation balloon via the inflow lumen based at least in part on the signal indicative of the blood flow sound and the second signal indicative of the second blood flow sound.
Example 15. The method of Example 14, further comprising: determining whether the signal indicative of the blood flow sound indicates a back flow proximal to the balloon; determining whether the second blood flow sound indicates substantially no flow distal to the balloon; and responsive to determining that the signal indicative of the blood flow sound indicates a back flow proximal to the cryoablation balloon and the second signal indicative of the second blood flow sound indicates substantially no flow distal to the cryoablation balloon, controlling the pump to decrease an inflation pressure for the balloon.
Example 16. A balloon catheter comprising an elongate body; a balloon coupled to the elongate body; an inflow lumen configured to provide a fluid to the balloon; and a microphone sensor coupled to the elongate body proximal of the balloon. The microphone sensor is configured to sense a flow of blood proximal to the balloon.
Example 17. The balloon catheter of Example 16, further comprising a second microphone sensor coupled to the elongate body distal of the balloon, wherein the second microphone is configured to sense a flow of blood distal to the balloon
Example 18. The balloon catheter of Example 15, further comprising: a second microphone sensor positioned to sense a flow of blood distal to the balloon.
Example 19. The balloon catheter of Example 16, wherein the microphone sensor is a piezoelectric microphone.
Example 20. The balloon catheter of Example 16, wherein the fluid is one selected from the group consisting of nitrous oxide, carbon dioxide, nitrogen, and argon.

Various features and advantages of the embodiments presented herein are set forth in the following claims.

What is claimed is:

1. A system comprising:
an electronic controller configured to:
couple to a balloon catheter comprising a balloon, an inflow lumen for providing a fluid to the balloon, and a microphone sensor is positioned to sense a flow of blood proximal to the balloon;
receive, from the microphone sensor, a signal indicative of a blood flow sound; and
control a pump to provide a volume of the fluid to the balloon via the inflow lumen based at least in part on the signal indicative of the blood flow sound.

2. The system of claim 1, further comprising:
the balloon catheter; and
the pump.

3. The system of claim 1, wherein the electronic controller is configured to:
perform signal processing on the signal indicative of the blood flow sound to produce a processed blood flow sound;
determine, based at least in part on the processed blood flow sound, a balloon occlusion percentage; and
control the pump to provide a volume of the fluid to the balloon via the inflow lumen based at least in part on the balloon occlusion percentage.

4. The system of claim 3, wherein the electronic controller is configured to:
when the balloon occlusion percentage meets a threshold, control the pump to maintain a current pressure in the balloon; and
when the balloon occlusion percentage does not meet the threshold, control the pump to increase an inflation pressure for the balloon.

5. The system of claim 1, wherein the electronic controller is configured to:
perform signal processing on the signal indicative of the blood flow sound to produce a processed blood flow sound;
determine, based at least in part on the processed blood flow sound, whether the balloon is over pressurized; and
based at least in part on determining that the balloon is over pressurized, control the pump to decrease an inflation pressure for the balloon.

6. The system of claim 1, wherein:
the balloon catheter includes a second microphone sensor positioned to sense a flow of blood distal to the balloon; and
the electronic controller is further configured to:
receive a second signal indicative of a second blood flow sound from the second microphone sensor, and
control the pump to provide the volume of the fluid to the balloon via the inflow lumen based at least in part on the signal indicative of the blood flow sound and the second signal indicative of the second blood flow sound.

7. The system of claim 6, wherein the electronic processor is configured to:
determine whether the signal indicative of the blood flow sound indicates a back flow proximal to the balloon;
determine whether the second signal indicative of the second blood flow sound indicates substantially no flow distal to the balloon; and
responsive to determining that the signal indicative of the blood flow sound indicates a back flow proximal to the balloon and the second signal indicative of the second blood flow sound indicates substantially no flow distal to the balloon, control the pump to decrease an inflation pressure for the balloon.

8. The system of claim 1, wherein the microphone sensor is a piezoelectric microphone.

9. The system of claim 1, wherein the microphone sensor is positioned outside of the balloon.

10. The system of claim 1, wherein the fluid is one selected from the group consisting of nitrous oxide, carbon dioxide, nitrogen, and argon.

11. A method comprising:
receiving, from a microphone sensor positioned to sense a flow of blood proximal to a balloon of a balloon catheter, a signal indicative of a blood flow sound; and
controlling a pump to provide a volume of fluid to the balloon based at least in part on the signal indicative of the blood flow sound.

12. The method of claim 11, further comprising:
performing signal processing on the signal indicative of the blood flow sound to produce a processed blood flow sound;
determining, from the processed blood flow sound, a balloon occlusion percentage; and
controlling the pump to provide a volume of the fluid to the cryoablation balloon via the inflow lumen based on the balloon occlusion percentage.

13. The method of claim 12, further comprising:
when the balloon occlusion percentage meets a threshold, controlling the pump to maintain a current pressure in the cryoablation balloon; and
when the balloon occlusion percentage does not meet the threshold, controlling the pump to increase an inflation pressure for the cryoablation balloon.

14. The method of claim 12, further comprising:
receiving a second signal indicative of a second blood flow sound from the second microphone sensor positioned to sense a flow of blood distal to the cryoablation balloon, and
controlling the pump to provide the volume of the fluid to the cryoablation balloon via the inflow lumen based at least in part on the signal indicative of the blood flow sound and the second signal indicative of the second blood flow sound.

15. The method of claim 14, further comprising:
determining whether the signal indicative of the blood flow sound indicates a back flow proximal to the balloon;
determining whether the second blood flow sound indicates substantially no flow distal to the balloon; and
responsive to determining that the signal indicative of the blood flow sound indicates a back flow proximal to the cryoablation balloon and the second signal indicative of the second blood flow sound indicates substantially no flow distal to the cryoablation balloon, controlling the pump to decrease an inflation pressure for the balloon.

16. A balloon catheter comprising:
an elongate body;
a balloon coupled to the elongate body;
an inflow lumen configured to provide a fluid to the balloon; and
a microphone sensor coupled to the elongate body proximal of the balloon,
wherein the microphone sensor is configured to sense a flow of blood proximal to the balloon.

17. The catheter of claim 16, further comprising:
a second microphone sensor coupled to the elongate body distal of the balloon, wherein the second microphone is configured to sense a flow of blood distal to the balloon.

18. The catheter of claim 16, wherein the microphone sensor is a piezoelectric microphone.

19. The catheter of claim 16, wherein the fluid comprises at least one selected from the group consisting of nitrous oxide, carbon dioxide, nitrogen, and argon.
